(19) United States
(12) Patent Application Publication
(10) Pub. No.: US 2020/0046476 A1
(43) Pub. Date: Feb. 13, 2020

(54) Ultrasonic Phototherapeutic Dental Braces And Use Method

(71) Applicant: Chinese Academy of Medical Sciences & Peking Union Medical College, Institute of Biomedical Engineer, Tianjin (CN)

(72) Inventors: Yingxin Li, Tianjin (CN); Xiaoxi Dong, Tianjin (CN); Zhaohui Zou, Tianjin (CN)

(73) Assignee: Chinese Academy of Medical Sciences & Peking Union Medical College, Institute of Biomedical Engineer, Tianjin (CN)

(21) Appl. No.: 16/357,403

(22) Filed: Mar. 19, 2019

(30) Foreign Application Priority Data
Aug. 13, 2018 (CN) 201810914069.3

(51) Int. Cl. A61C 17/32; A61C 17/20; A61C 19/06; A61N 5/06; A61C 7/08

(52) U.S. Cl. CPC A61C 17/32; A61C 17/20; A61C 19/06; A61N 5/0603; A61N 2005/067; A61N 2005/0606; A61N 2005/063; A61N 2005/0651; A61C 7/08

(57) ABSTRACT

The present invention discloses ultrasonic phototherapeutic dental braces and a use method. The dental braces include: a transparent substrate, a phototherapeutic optical fiber, an ultrasonic transducer, an incoming line slot, a wire, and a plastic keel, where the phototherapeutic optical fiber, the ultrasonic transducer, the wire, and the plastic keel are all disposed inside the transparent substrate; the transparent substrate includes an arched end and a performed slot; the performed slot is used for fastening the transparent substrate on teeth of a person; the incoming line slot is disposed at the arched end of the transparent substrate; the incoming line slot is used for leading the wire and the phototherapeutic optical fiber into the transparent substrate; the phototherapeutic optical fiber is used for emitting therapy light to irradiate the therapy light into the oral cavity; the wire is connected to the ultrasonic transducer, the wire is externally connected to a power supply, and is used for supplying power for the ultrasonic transducer; the ultrasonic transducer is used for emitting ultrasonic waves; and the plastic keel is used for adjusting a shape of the transparent substrate. The present invention harmonically combines mechanically cleaning the oral cavity with ultrasonic waves with a photobiomodulation on the oral cavity, and can better protect the oral cavity environment of a user.

This application claims priority to Chinese application number 201810914069.3, filed 13 Aug. 2018 with a title of ULTRASONIC PHOTOTHERAPY MOUTHPIECE AND USE METHOD. The above-mentioned patent application is incorporated herein by reference in its entirety.

### TECHNICAL FIELD

The present invention relates to the field of dental braces, and in particular, to ultrasonic phototherapeutic dental braces and a use method.

### BACKGROUND

Brushing teeth with a toothbrush is a common oral cleaning method. Shapes of existing toothbrushes and bristles are lack of specialized design, and cannot fully satisfy requirements of cleaning all tooth profiles and thoroughly cleaning up calculus, plaque, and pigment on a surface of a tooth, a corner of a tooth, and a tiny slit between teeth, and there is also a problem that improper use causes damage to teeth, gums, teethridges, and the like. Chemical ingredients of toothpaste used during teeth brushing also bring a risk of a disadvantageous impact on the body. Bristles of existing electric toothbrushes are electrically driven to rapidly repeatedly brush teeth. Although cleaning efficiency can be improved, a problem that improper use causes damage to teeth, gums, teethridges, and the like is severer. Although existing ultrasonic dental braces can effectively clean a surface of oral tissue, it is difficult for the ultrasonic dental braces to implement plaque control against deeper lesions (for example, periodontal pockets) in the oral cavity, to diminish inflammation and ease pain, and to promote wound healing. In addition, because there are dark substances, such as dental stains, dental calculus, as well as plaque and pigmentation, having an effect of strongly absorbing therapy light in the oral cavity of a patient, it is difficult for the existing phototherapeutic dental braces to perform irradiation treatment on a corner of a tooth and a tiny slit between teeth that are formed below the substances and that cannot be irradiated by therapy light.

### SUMMARY

With regard to the foregoing problems, the present invention provides ultrasonic phototherapeutic dental braces and a use method.

To achieve the above purpose, the present invention provides the following solution:

Ultrasonic phototherapeutic dental braces are provided, including: a transparent substrate, a phototherapeutic optical fiber, an ultrasonic transducer, an incoming line slot, a wire, and a plastic keel, where the phototherapeutic optical fiber, the ultrasonic transducer, the wire, and the plastic keel are all disposed inside the transparent substrate; the transparent substrate includes an arched end and a performed slot; the performed slot is used for fastening the transparent substrate on teeth of a person; the incoming line slot is disposed at the arched end of the transparent substrate; the incoming line slot is used for leading the wire and the phototherapeutic optical fiber into the transparent substrate; the phototherapeutic optical fiber is disposed on a sidewall of the transparent substrate, the phototherapeutic optical fiber is used for emitting therapy light to irradiate the therapy light into the oral cavity; the wire is connected to the ultrasonic transducer, and the wire is externally connected to a power supply, and is used for supplying power for the ultrasonic transducer; the ultrasonic transducer is located inside a base plate of the transparent substrate, the ultrasonic transducer is used for emitting ultrasonic waves; and the plastic keel is disposed inside the base plate of the transparent substrate, and the plastic keel is used for adjusting a shape of the transparent substrate.

Optionally, the shape of the transparent substrate matches a shape of the dental arch of a user.

Optionally, the transparent substrate is made of transparent soft material.

Optionally, the phototherapeutic optical fiber is an entire-surface light-emitting optical fiber or a partial-surface light-emitting optical fiber.

Optionally, the phototherapeutic optical fiber is externally connected to a light source through an optical fiber interface, and the light source is laser or LED.

Optionally, the plastic keel is made of metal material.

A use method of the above-described ultrasonic phototherapeutic dental braces is provided, including:

fastening a performed slot of a transparent substrate on teeth of a person;

supplying power to an ultrasonic transducer by using a wire;

oscillating, by the ultrasonic transducer, and generating ultrasonic waves;

transferring the ultrasonic waves into the oral cavity by using the transparent substrate;

leading the therapy light into a phototherapeutic optical fiber by using an external light source; and

irradiating, the phototherapeutic optical fiber, the therapy light into the oral cavity.

Compared with the prior art, the present invention has the following technical effects: the present invention harmonically combines mechanically cleaning the oral cavity with ultrasonic waves and photobiomodulation on the oral cavity, and compared with the conventional toothbrushes, electric toothbrushes, and existing dental braces having an ultrasonic cleaning function and phototherapeutic dental braces, the present invention can better protect the oral cavity environment of a user. Near a target irradiated area in the oral cavity, a refractive index of a cladding of the phototherapeutic optical fiber is accurately controlled to be close to or higher than that of a core layer of the optical fiber, to enable therapy light to transmit through the core layer to irradiate a surface of the target irradiated area in the oral cavity, thereby achieving an accurate phototherapeutic effect. The phototherapeutic optical fiber can achieve uniform light irradiation in the entire oral cavity, and can also achieve targeted irradiation on a specified area in the oral cavity. Because light has specific transmissivity in biological issue, irradiation can be implemented on a slit and deep tissue (for example, periodontal pockets) that can hardly be cleaned with the conventional toothbrushes, electric toothbrushes, and existing dental braces having an ultrasonic cleaning function, and such irradiation produces a photobiomodulation on the oral tissue, so as to produce effects of controlling plaque, diminishing inflammation and easing pain, and promoting wound healing. Ultrasonic energy generated by an ultrasonic transducer is transferred through a transparent substrate and a water environment inside the oral cavity of a patient, thereby effectively cleaning up dental stains, dental calculus, plaque and pigmentation, and the like attached to a surface of the oral issue such as teeth and gums.

### BRIEF DESCRIPTION OF THE DRAWINGS

To describe the technical solutions in the embodiments of the present invention or in the prior art more clearly, the following briefly describes the accompanying drawings required for describing the embodiments. Apparently, the accompanying drawings in the following description show some embodiments of the present invention, and a person of ordinary skill in the art may still derive other drawings from these accompanying drawings without creative efforts.

FIG. 1 is a schematic structural diagram of Ultrasonic phototherapeutic dental braces according to an embodiment of the present invention;

FIG. 2 is a cross-sectional view of Ultrasonic phototherapeutic dental braces according to an embodiment of the present invention; and

FIG. 3 is a flowchart of a use method of Ultrasonic phototherapeutic dental braces according to an embodiment of the present invention.

### DETAILED DESCRIPTION OF EMBODIMENTS

The following clearly and completely describes the technical solutions in the embodiments of the present invention with reference to the accompanying drawings in the embodiments of the present invention. Apparently, the described embodiments are merely a part rather than all of the embodiments of the present invention. All other embodiments obtained by a person of ordinary skill in the art based on the embodiments of the present invention without creative efforts shall fall within the protection scope of the present invention.

To make objectives, features, and advantages of the present invention more comprehensible, the following describes the present invention in more detail with reference to accompanying drawings and specific implementations.

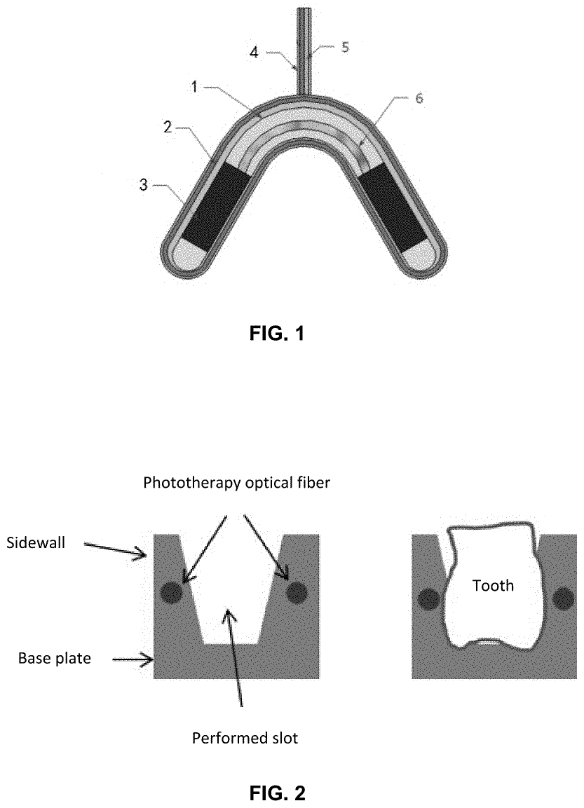

As shown in FIG. 1, ultrasonic phototherapeutic dental braces include: a transparent substrate 1, a phototherapeutic optical fiber 2, an ultrasonic transducer 3, an incoming line slot 4, a wire 5, and a plastic keel 6. The phototherapeutic optical fiber 2, the ultrasonic transducer 3, the wire 5, and the plastic keel 6 are all disposed inside the transparent substrate 1.

A shape of the transparent substrate 1 matches a shape of the dental arch of a person, and the transparent substrate 1 includes an arched end and a performed slot, and is made of medical or food-grade transparent soft material such as silica gel or resin. The performed slot is used for fastening the transparent substrate on teeth of a person. As shown in FIG. 2, during use, teeth of a patient are placed in the performed slot of the transparent substrate 1, to enable the soft material of the transparent substrate to deform to perfectly match the shape of the teeth of the patient. The incoming line slot 4 is disposed at the arched end of the transparent substrate 1. The incoming line slot 4 is used for leading the wire 5 and the phototherapeutic optical fiber 2 into the transparent substrate 1. The phototherapeutic optical fiber is disposed on a sidewall of the transparent substrate, and the phototherapeutic optical fiber is used for emitting therapy light to irradiate the therapy light into the oral cavity, to implement internal and external irradiation on the teeth. Near a target irradiated area in the oral cavity, a refractive index of a cladding of the phototherapeutic optical fiber is accurately controlled to be close to or higher than that of a core layer of the optical fiber, to enable therapy light to transmit through the core layer to irradiate a surface of the target irradiated area in the oral cavity, thereby achieving an accurate phototherapeutic effect. The wire is connected to the ultrasonic transducer, and the wire is externally connected to a power supply, and is used for supplying power for the ultrasonic transducer. The ultrasonic transducer is used for emitting ultrasonic waves, the ultrasonic transducer is located inside the transparent substrate base plate, and its size does not affect light emission and irradiation of the phototherapeutic optical fiber. A plastic keel (usually made of metal material) is also reserved in the base plate, and the shape (mainly, the size of a dental arch angle) of the dental arch of the transparent substrate is adjusted according to the size of the oral cavity of the patient. The phototherapeutic optical fiber is an entire-surface light-emitting optical fiber or a partial-surface light-emitting optical fiber. That is, near a target irradiated area in the oral cavity, a refractive index of a cladding of the phototherapeutic optical fiber is accurately controlled to be close to or higher than that of a core layer of the optical fiber, to enable therapy light to transmit through the core layer to irradiate a surface of the target irradiated area in the oral cavity, thereby achieving an accurate phototherapeutic effect. When therapy light is lead in, the entire optical fiber or a specific area of the optical fiber emits light, so that the optical fiber is used as a light source for implementing phototherapy, and a terminal thereof is connected to an external light source (usually, laser or an LED) through a standard optical fiber interface (for example, the SMA905 interface or the FC interface).

According to the specific embodiments provided by the present invention, the present invention discloses the following technical effects: the present invention harmonically combines mechanically cleaning the oral cavity with ultrasonic waves with photobiomodulation on the oral cavity, and compared with the conventional toothbrushes, electric toothbrushes, and existing dental braces having an ultrasonic cleaning function and phototherapeutic dental braces, the present invention can better protect the oral cavity environment of a user. Near a target irradiated area in the oral cavity, a refractive index of a cladding of the phototherapeutic optical fiber is accurately controlled to be close to or higher than that of a core layer of the optical fiber, to enable therapy light to transmit through the core layer to irradiate a surface of the target irradiated area in the oral cavity, thereby achieving accurate photobiomodulation. The phototherapeutic optical fiber can achieve uniform light irradiation in the entire oral cavity, and can also achieve targeted irradiation on a specified area in the oral cavity. Because light has specific transmissivity in biological issue, irradiation can be implemented on a slit and deep tissue (for example, periodontal pockets) that can hardly be cleaned with the conventional toothbrushes, electric toothbrushes, and existing dental braces having an ultrasonic cleaning function, and such irradiation produces a photobiomodulation on the oral tissue, so as to produce effects of controlling plaque, diminishing inflammation and easing pain, and promoting wound healing. Ultrasonic energy generated by an ultrasonic transducer is transferred through a transparent substrate and a water environment inside the oral cavity of a patient, thereby effectively cleaning up dental stains, dental calculus, plaque and pigmentation, and the like attached to a surface of the oral issue such as teeth and gums.

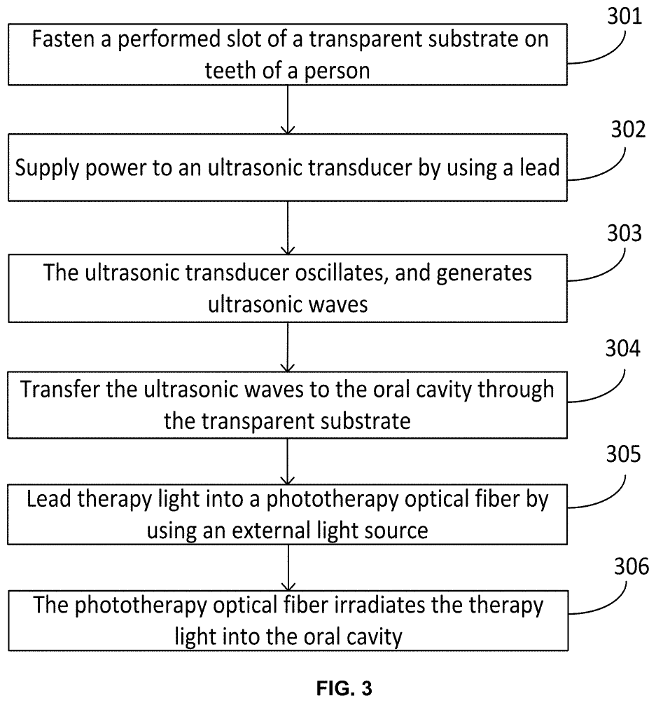

As shown in FIG. 3, a use method of the foregoing ultrasonic phototherapeutic dental braces include:

Step 301: Fasten a performed slot of a transparent substrate on teeth of a person.

Step 302: Supply power to an ultrasonic transducer by using a wire.

Step 303: The ultrasonic transducer oscillates, and generates ultrasonic waves.

Step 304: Transfer the ultrasonic waves to the oral cavity through the transparent substrate.

Step 305: Lead therapy light into a phototherapeutic optical fiber by using an external light source.

Step 306: The phototherapeutic optical fiber irradiates the therapy light into the oral cavity.

A doctor applies an external force to the dental braces according to a shape of the oral cavity of a patient, to make a shape of the dental braces to fit the oral cavity of the patient. After the patient wears the dental braces, the patient holds water in the mouth as a transfer medium of ultrasonic energy. Power is supplied for the ultrasonic transducer by using the wire, the ultrasonic transducer oscillates, and generates ultrasonic waves, and the ultrasonic waves are transferred by the substrate of the transparent dental braces to the oral cavity, to clean up dental stains, dental calculus, plaque and pigmentation, and the like attached to issue such as teeth and gums. The therapy light is lead into the phototherapeutic optical fiber by using the external light source, to implement light regulation therapy in the entire mouth. The ultrasonic therapy and phototherapeutic can be carried out alternately according to an oral cavity status of a patient, so as to clean the oral cavity of the patient.

Each embodiment of the present specification is described in a progressive manner, each embodiment focuses on the difference from other embodiments, and the same and similar parts between the embodiments may refer to each other.

Several examples are used for illustration of the principles and implementation methods of the present invention. The description of the embodiments is used to help illustrate the method and its core principles of the present invention. In addition, those skilled in the art can make various modifications in terms of specific embodiments and scope of application in accordance with the teachings of the present invention. In conclusion, the content of this specification shall not be construed as a limitation to the invention.

What is claimed is:

1. Ultrasonic phototherapeutic dental braces, comprising: a transparent substrate, a phototherapeutic optical fiber, an ultrasonic transducer, an incoming line slot, a wire, and a plastic keel, wherein the phototherapeutic optical fiber, the ultrasonic transducer, the wire, and the plastic keel are all disposed inside the transparent substrate; the transparent substrate comprises an arched end and a performed slot; the performed slot is used for fastening the transparent substrate on teeth of a person; the incoming line slot is disposed at the arched end of the transparent substrate; the incoming line slot is used for leading the wire and the phototherapeutic optical fiber into the transparent substrate; the phototherapeutic optical fiber is disposed on a sidewall of the transparent substrate, the phototherapeutic optical fiber is used for emitting therapy light to irradiate the therapy light into the oral cavity; the wire is connected to the ultrasonic transducer, and the wire is externally connected to a power supply, and is used for supplying power for the ultrasonic transducer; the ultrasonic transducer is located inside a base plate of the transparent substrate, the ultrasonic transducer is used for emitting ultrasonic waves; and the plastic keel is disposed inside the base plate of the transparent substrate, and the plastic keel is used for adjusting a shape of the transparent substrate.

2. The dental braces according to claim 1, wherein the shape of the transparent substrate matches a shape of the dental arch of a person.

3. The dental braces according to claim 1, wherein the transparent substrate is made of transparent soft material.

4. The dental braces according to claim 1, wherein the phototherapeutic optical fiber is an entire-surface light-emitting optical fiber or a partial-surface light-emitting optical fiber.

5. The dental braces according to claim 1, wherein the phototherapeutic optical fiber is externally connected to a light source through an optical fiber interface, and the light source is laser or an LED.

6. The dental braces according to claim 1, wherein the plastic keel is made of metal material.

7. A use method of the ultrasonic phototherapeutic dental braces according to claim 1, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.

8. A use method of the ultrasonic phototherapeutic dental braces according to claim 2, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.

9. A use method of the ultrasonic phototherapeutic dental braces according to claim 3, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.

10. A use method of the ultrasonic phototherapeutic dental braces according to claim 4, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.

11. A use method of the ultrasonic phototherapeutic dental braces according to claim 5, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.

12. A use method of the ultrasonic phototherapeutic dental braces according to claim 6, wherein the use method comprises:
fastening a performed slot of a transparent substrate on teeth of a person;
supplying power to an ultrasonic transducer by using a wire;
oscillating, by the ultrasonic transducer, and generating ultrasonic waves;
transferring the ultrasonic waves into the oral cavity by using the transparent substrate;
leading the therapy light into a phototherapeutic optical fiber by using an external light source; and
irradiating, the phototherapeutic optical fiber, the therapy light into the oral cavity.
